(19) United States
(12) Patent Application Publication
(10) Pub. No.: US 2014/0186829 A1
(43) Pub. Date: Jul. 3, 2014

(54) METHOD FOR PREDICTING INSULINOPENIC TYPE 2 DIABETES

(71) Applicants: Remy Burcelin, Toulouse (FR); Jacques Amar, Toulouse (FR); Beverly Balkau, Villejuif (FR)

(72) Inventors: Remy Burcelin, Toulouse (FR); Jacques Amar, Toulouse (FR); Beverly Balkau, Villejuif (FR)

(73) Assignees: CHU De Toulouse, Toulouse Cedex 9 (FR); INSTITUT NATIONAL DE LA SANTE ET DE LA RECHERCHE MEDICALE (INSERM), Paris (FR)

(21) Appl. No.: 14/008,631

(22) PCT Filed: Apr. 2, 2012
(86) PCT No.: PCT/EP2012/055933

§ 371 Date: Dec. 6, 2013

(30) Foreign Application Priority Data
Mar. 31, 2011 (EP) 11305367.2

(51) Int. Cl. C12Q 1/68

(52) U.S. Cl. CPC C12Q 1/689
USPC 435/6.11; 435/6.15; 435/6.12

(57) ABSTRACT

The present invention relates to an in vitro method, for predicting a risk of onset of type 2 diabetes in a subject, which method comprises the steps of: a) measuring the concentration of bacterial 16S rDNA in a biological sample of said subject; and b) comparing said measured concentration of bacterial 16S rDNA to a threshold level; wherein a measured concentration of bacterial 16S rDNA higher than the threshold level is indicative of an increased risk of onset of type 2 diabetes in the subject, and a measured concentration of bacterial 16S rDNA lower than the threshold level is indicative of a decreased risk of onset of type 2 diabetes in the subject.

The present invention concerns a method for predicting type 2 diabetes with predominant insulinopenia.

The incidence of diabetes, and in particular of type 2 diabetes, both in developed and emerging countries has reached epidemic proportions. Indeed, the World Health Organization has predicted that the number of diabetics will double from 143 million in 1997 to about 300 million in 2025, largely because of dietary and other lifestyle factors. Therefore, it is of utmost importance to detect subjects at risk of metabolic diseases at an early stage, when slight lifestyle modifications may be efficient and easier to install.

In this respect, the current recognized risk factors for diabetes, such as central adiposity or high fasting blood glucose, are markers of an advanced stage of metabolic disease. Furthermore, there is a need for markers of diabetes risk that are independent of metabolic features in order to generate new concepts for therapeutic strategies. This need is illustrated by the lack of a clear demonstration of the beneficial effect of tight blood glucose control on macroangiopathy after a decade of well-designed randomized trials (Dormandy et al. (2005) Lancet 366:1279-1289; UK Prospective Diabetes Study Group (1998) Lancet 352:837-853; Action to control cardiovascular risk in diabetes Study Group (2008) N. Engl. J. Med. 358:2545-2559; Group et al. (2008) N. Engl. J. Med. 358:2560-2572; Duckworth et al. (2009) N. Engl. J. Med. 360:129-139).

From a pathophysiological point of view, experimental data have already linked weight and the metabolic syndrome with gut microbiota, and innate immunity against infectious diseases (Turnbaugh et al. (2009) Nature 457:480-484: Cani et al. (2007) Diabetes 56:1761-1772). The inventors and others have demonstrated in animal models the influence of gut microbiota and, in particular, of blood gram negative bacteria, on body weight and metabolic disease. In humans, periodontitis, a chronic gram negative infectious disease of the oral cavity was associated with the metabolic syndrome and insulin resistance in cross-sectional studies (Sabbah et al. (2008) J. Clin. Endocrinol. Metab. 93:3989-3994; Benguigui et al. (2010) J. Clin. Periodontol. 37:601-608). Nevertheless, these studies did not enable identifying early markers of diabetes.

Type 2 diabetes is the result of the resistance of the tissue targets towards insulin action and of a relative insulinopenia due to an affection of pancreatic beta cells. It variably associates either (i) a predominant insulin resistance with a moderate insulinopenia, or (ii) a moderate insulin resistance with a predominant insulinopenia. Currently, the screening of patients at risk of diabetes is based on markers of an advanced stage of the disease, when both insulin resistance and insulinopenia are already present. Nevertheless, different therapeutical families of compounds differentially target insulin resistance or insulinopenia. Accordingly, the early detection of patients at risk of developing type 2 diabetes with regards to insulinopenia or insulin resistance could lead to targeted preventing treatments. There is thus a need for identifying new markers enabling the early detection of patients at risk of type 2 diabetes with predominant insulin resistance or with predominant insulinopenia.

The present invention first arises from the unexpected finding by the inventors that the plasma concentration of bacterial 16S rDNA predicted the onset of type 2 diabetes, in a large cohort of apparently healthy subjects, independently of traditional metabolic risk factors. The present invention also arises from the finding by the inventors that the plasma concentration of bacterial 16S rDNA was a marker for the very early prediction of pancreatic beta cell failure, and enabled excluding with a very high probability the onset of type 2 diabetes with insulinopenia in patients at risk of diabetes.

The present invention thus relates to a method, in particular an in vitro method, for predicting a risk of onset of type 2 diabetes in a subject, which method comprises the steps of:

a) measuring the concentration of bacterial 16S rDNA in a biological sample of said subject; and

b) comparing said measured concentration of bacterial 16S rDNA to a threshold level;

wherein a measured concentration of bacterial 16S rDNA higher than the threshold level is indicative of an increased risk of onset of type 2 diabetes in the subject, and a measured concentration of bacterial 16S rDNA lower than the threshold level is indicative of a decreased risk of onset of type 2 diabetes in the subject.

### DETAILED DESCRIPTION OF THE INVENTION

### Diabetes

As used herein, “diabetes” or “diabetes mellitus” denotes a metabolic disorder in which the pancreas produces insufficient amounts of insulin, or in which the cells of the body fail to respond appropriately to insulin thus preventing cells from absorbing glucose. As a result, glucose builds up in the blood. This high blood glucose level produces the classical symptoms of polyuria (frequent urination), polydipsia (increased thirst) and polyphagia (increased hunger). The term “diabetes” includes type 1 diabetes, type 2 diabetes, gestational diabetes (during pregnancy) and other states that cause hyperglycaemia.

Type 1 diabetes, also called insulin-dependent diabetes mellitus (IDDM) and juvenile-onset diabetes, is caused by β-cell destruction, usually leading to absolute insulin deficiency.

Type 2 diabetes, also known as non-insulin-dependent diabetes mellitus (NIDDM) and adult-onset diabetes, is associated with predominant insulin resistance and thus relative insulin deficiency and/or a predominantly insulin secretory defect (or insulinopenia) with insulin resistance. More specifically, type 2 diabetes may be associated either with (i) a predominant insulin resistance with a moderate insulinopenia or with (ii) a moderate insulin resistance with a predominant insulinopenia.

In the context of the invention, the expression “insulin resistance” refers to a physiological condition where the natural hormone, insulin, becomes less effective at lowering blood sugars. The resulting increase in blood glucose may raise levels outside the normal range and cause adverse health effects.

In the context of the invention, the term “insulinopenia” or “insulin deficiency” refers to an insufficient production of insulin in view of the needs of the subject. This insufficient production may be due to kinetic and quantitative or qualitative defaults. In particular, it may be due to an insufficient maximal secretory capacity of β-cells in response to glucose stimuli. It may also be due to defaults in the insulin maturation from the proinsulin pro-hormone.

“Pancreatic beta cells”, “beta-cells” or “β-cells” are a type of cell in the pancreas in areas called the islets of Langerhans, which makes and releases insulin. Apart from insulin, β cells release C-peptide, a by-product of insulin production, into the bloodstream in equimolar quantities. C-peptide helps to prevent neuropathy and other symptoms of diabetes related to vascular deterioration. β cells also produce amylin, also known as IAPP, islet amyloid polypeptide, which functions as part of the endocrine pancreas and contributes to glycemic control.

In the context of the invention, the expression “pancreatic β cell failure” refers to a disorder of the above defined β cells, which decreases, inhibits and/or stops their capacity of producing insulin.

As used herein, the expression “hepatic cytolysis” refers to the lysis of hepatic cells.

### Subject

In the context of the present invention, a “subject” denotes a human or non-human mammal, such as a rodent (rat, mouse, rabbit), a primate (chimpanzee), a feline (cat), or a canine (dog). Preferably, the subject is human. The subject according to the invention may be in particular a male or a female.

Preferably, the subject according to the invention is 30-65 year old.

In a particular embodiment of the invention, the subject is at risk of diabetes.

As used herein, the expression “subject at risk of diabetes” refers to a subject as defined above who has an increased likelihood of developing diabetes as defined above. In particular, a subject at risk of diabetes according to the invention is a subject who displays at least one known diabetes risk factor.

As used herein, the expression “diabetes risk factor” refers to a biological marker which is associated with the onset of diabetes. Some diabetes risk factors are well-known from the skilled person and include for example age (greater than 45 years), diabetes during a previous pregnancy, excess body weight, especially central adiposity, family history of diabetes, smoking, given birth to a baby weighing more than 9 pounds, low HDL cholesterol, high blood levels of triglycerides, high fasting glycemia, high blood pressure or hypertension, impaired glucose tolerance, low activity level, metabolic syndrome and polycystic ovarian syndrome.

In the context of the invention, the expression “central adiposity” or “high weight circumference” are used indifferently and refers to an accumulation of abdominal fat resulting in an increase in waist size or circumference. While central obesity can be obvious just by looking at the naked body, the severity of central obesity is determined by taking waist and hip measurements. The absolute waist circumference (>102 centimeters (40 inches) in men and >88 centimeters (35 inches) in women) and the waist-hip ratio (>0.9 for men and >0.85 for women) are both used as measures of central obesity. Preferably, the expression “central adiposity” according to the invention refers to a waist circumference of more than 102 cm in men or of more than 88 cm in women.

As used herein, the expression “family history of diabetes” refers to the presence of at least one case of diabetes, in particular of type 2 diabetes, among the family of the subject, in particular among its ascendants (father, mother) and its siblings.

As used herein, the expression “low HDL cholesterol” refers to a blood level of HDL (high density lipoprotein) cholesterol inferior to 40 mg/dl in men and 50 mg/dl in women.

As used herein, the expression “high blood levels of triglycerides” refers to a blood level of triglycerides superior to 250 mg/dl.

As used herein, “high fasting glycemia” denotes a syndrome of disordered metabolism, resulting in a glycemia, in particular a fasting glycemia, of more than 6.1 mmol/I.

As used herein, “hypertension”, also referred to as “high blood pressure”, “HTN” or “HPN”, denotes a medical condition in which the blood pressure is chronically elevated. In the context of the invention, hypertension is preferably defined by systolic/diastolic blood pressure of at least 140/90 mmHg or being on antihypertensive medication.

As used herein, the expression “impaired glucose tolerance” refers to a pre-diabetic state of dysglycemia that is associated with insulin resistance and increased risk of cardiovascular pathology. In particular, impaired glucose tolerance is defined as two-hour glucose levels of 140 to 199 mg/dL (7.8 to 11.0 mmol) on the 75-g oral glucose tolerance test. A patient is said to be under the condition of impaired glucose tolerance when he/she has an intermediately raised glucose level after 2 hours, but less than would qualify for type 2 diabetes mellitus. The fasting glucose may be either normal or mildly elevated.

As used herein, the expression “low activity level” refers to the fact of exercising less than 3 times a week.

As used herein, the expression “metabolic syndrome” refers to a multiplex risk factor for cardiovascular disease comprising the 6 following components: abdominal obesity, atherogenic dyslipidemia, raised blood pressure, insulin resistance with or without glucose intolerance, proinflammatory state and prothrombotic state. The metabolic syndrome is more specifically defined in Grundy et al. (2004) Circulation 109:433-438.

As used herein, the expression “polycystic ovarian syndrome”, “PCOS”, “polycystic ovary disease”, “PCOD”, “functional ovarian hyperandrogenism”, “ovarian hyperthecosis” or “sclerocystic ovary syndrome” refers to a female endocrine disorder defined by the presence of oligoovulation and signs of androgen excess.

Preferably, the subject according to the invention displays at least one diabetes risk factor selected from the group consisting of:
a waist circumference of more than 102 cm in men or of more than 88 cm in women;
smoking;
a fasting glycemia equal or superior to 6.1 mmol/L;
hypertension; and
family history of diabetes.

In another preferred embodiment, the subject according to the invention is on the contrary free of central adiposity, high fasting glycemia or of the metabolic syndrome.

In another preferred embodiment, the subject according to the invention is free of bacteremia. Accordingly, the subject according to the invention preferably displays a plasma baseline C reactive protein concentration lower than 30 mg/l.

As used herein, the term “C reactive protein” or “CRP” refers to a protein which is a member of the class of acute-phase reactants, as its levels rise dramatically during inflammatory processes occurring in the body. As known from the skilled person, CRP is a 224-residue protein with a monomer molar mass of 25106 Da, encoded by the CRP gene.

### Bacterial 16S rDNA

In the context of the invention, the expressions “16S rDNA” or “16S ribosomal DNA” are used indifferently and refer to the gene encoding the 16S ribosomal RNA constituted of about 1500 nucleotides, which is the main component of the small prokaryotic ribosomal subunit (30S). 16S rDNA is highly conserved among bacteria. The reference Escherichia coli 16S rDNA gene sequence corresponds to SEQ ID NO: 1 (called rrsA). In the context of the invention, 16S rDNA refers to any sequence corresponding to SEQ ID NO: 1 in other bacterial strains.

### In Vitro Method for Predicting

The present invention concerns an in vitro method for predicting a risk of onset of type 2 diabetes in a subject, which method comprises the steps of:

a) measuring the concentration of bacterial 16S rDNA in a biological sample of said subject; and

b) comparing said measured concentration of bacterial 16S rDNA to a threshold level;

wherein a measured concentration of bacterial 16S rDNA higher than the threshold level is indicative of an increased risk of onset of type 2 diabetes in the subject, and a measured concentration of bacterial 16S rDNA lower than the threshold level is indicative of a decreased risk of onset of type 2 diabetes in the subject.

As used herein, a “predicting method” or “method for predicting” refers to a method for determining whether an individual is likely to develop a disease.

As used herein, the expression “risk of onset” of a disease refers to the probability that a disease will appear in a studied subject, in particular within a given period of time.

Preferably, the concentration of bacterial 16S rDNA is measured by polymerase chain reaction (PCR), more preferably by quantitative PCR (qPCR), most preferably by real-time or real-time quantitative PCR (RT-PCR or RT-qPCR).

As used herein, “real-time PCR”, “real-time quantitative PCR”, “real-time polymerase chain reaction” or “kinetic polymerase chain reaction” refers to a laboratory technique based on the polymerase chain reaction, which is used to amplify and simultaneously quantify a targeted DNA molecule. It enables both detection and quantification (as absolute number of copies or relative amount when normalized to DNA input or additional normalizing genes) of a specific sequence in a sample. Two common methods of quantification are the use of fluorescent dyes that intercalate with double-stranded DNA, and modified DNA oligonucleotide probes that fluoresce when hybridized with a complementary DNA.

As used herein, the term “biological sample” means a substance of biological origin. Examples of biological samples include, but are not limited to, blood and components thereof such as serum, plasma, platelets, subpopulations of blood cells such as leucocytes, urine, and tissues such as adipose tissues, hepatic tissues, pancreatic tissues and the like. Preferably, a biological sample according to the present invention is a blood, serum, plasma, leucocytes, urine, adipose tissue or hepatic tissue sample. More preferably, the biological sample is selected from the group consisting of blood, serum and plasma sample. The biological sample according to the invention may be obtained from the subject by any appropriate means of sampling known from the skilled person.

The method of predicting diabetes according to the invention comprises a step of comparing the measured concentration of bacterial 16S rDNA to a threshold level.

Preferably, the threshold level corresponds to the normal level of bacterial 16S rDNA.

As intended herein a “normal level” of bacterial 16S rDNA means that the concentration of 16S rDNA in the biological sample is within the norm cut-off values for that gene. The norm is dependant on the biological sample type and on the method used for measuring the concentration of 16S rDNA in the biological sample. In particular, the threshold level is the mean level of concentration of 16S rDNA in a healthy population.

As used herein, a “healthy population” means a population constituted of subjects who have not previously been diagnosed with diabetes or who do not display diabetes risk factors as defined above. Subjects of a healthy population also do not otherwise exhibit symptoms of disease. In other words, such subjects, if examined by a medical professional, would be characterized as healthy and free of symptoms of disease.

The threshold level may also be the level of concentration of 16S rDNA measured in a group corresponding to the highest deciles, preferably the eighth decile, of a population predicted to be at risk of diabetes.

Preferably, in the methods according to the invention, the threshold level is between 0.5 and 0.25 ng/A, still preferably between 0.10 and 0.20 ng/A, most preferably of 0.15 ng/μL of bacterial 16S rDNA, said threshold level being preferably used when the concentration of bacterial 16S rDNA is measured by real-time PCR, preferably using the universal forward and reverse primers eubac-F (5′-TCCTACGGGAGGCAGCAGT-3′ SEQ ID NO: 2) and eubac-R (5′-GGACTACCAGGGTATCTAATCCTGTT-3′ SEQ ID NO: 3), typically using the following reaction conditions for amplification of DNA: 95° C. for 10 min and 35 cycles of 95° C. for 15 s and 60° C. for 1 min.

Preferably, in the methods of the invention, it is further determined whether the measured concentration of bacterial 16S rDNA is increased or decreased compared to the threshold level according to the invention.

Preferably, when the measured concentration of bacterial 16S rDNA is increased compared to the threshold level, its level is significantly higher than the threshold level.

Preferably, when the measured concentration of bacterial 16S rDNA is decreased compared to the threshold level, its level is significantly lower than the threshold level.

The inventors specifically demonstrated that the presence of a lower concentration of bacterial 16S rDNA in the biological sample of a subject compared to the threshold level enabled excluding with a very high significance the onset of type 2 diabetes with predominant insulinopenia.

As will be easily understood by the skilled person, this specific association between a low concentration of bacterial 16S rDNA and a decreased risk of onset of type 2 diabetes with predominant insulinopenia cannot be applied in a reverse way. In other words, the inventors did not demonstrate that a higher concentration of bacterial 16S rDNA in the biological sample of a subject compared to the threshold level would be indicative of a higher risk of onset of type 2 diabetes with predominant insulinopenia.

Accordingly, preferably, in the methods according to the invention, a measured concentration of bacterial 16S rDNA in the biological sample of the subject which is lower than the threshold level is indicative of a decreased risk of onset of type 2 diabetes with predominant insulinopenia.

On the contrary, the inventors demonstrated that the presence of a higher concentration of bacterial 16S rDNA in the biological sample of a subject compared to the threshold level was indicative of pancreatic beta cell failure.

Accordingly, preferably, in the methods according to the invention, a measured concentration of bacterial 16S rDNA higher than the threshold level is further predictive of pancreatic beta cell failure.

Moreover, the inventors demonstrated that the concentration of bacterial 16S rDNA enabled predicting the onset of type 2 diabetes as soon as 9 years before the onset of the disease.

Accordingly, in preferred methods according to the invention, a measured concentration of bacterial 16S rDNA in the biological sample of the subject which is lower than the threshold level is indicative of a decreased risk of onset of type 2 diabetes with predominant insulinopenia within 9 years from the sampling, more particularly within a period of 6 to 9 years from the sampling.

The present inventors further demonstrated that the concentration of bacterial 16S rDNA in the plasma of a subject was also associated with the level of alanine transaminase (ALT), which is a biological marker of hepatic cytolysis.

Accordingly, the present invention also relates to an in vitro method for predicting a risk of hepatic cytolysis in a subject, which method comprises the steps of:

a) measuring the concentration of bacterial 16S rDNA in a biological sample of said subject as defined above; and

b) comparing said measured concentration of bacterial 16S rDNA to a threshold level as defined above;

wherein a measured concentration of bacterial 16S rDNA higher than the threshold level is indicative of an increased risk of hepatic cytolysis.

### Treatment Regimen

The inventors demonstrated that the concentration of bacterial 16S rDNA was a predictive marker enabling excluding the onset of type 2 diabetes with predominant insulinopenia. Since treatment regimens currently used to treat type 2 diabetes specifically target either pancreatic beta cell failure or insulin resistance, this marker also enables determining whether a subject suffering from type 2 diabetes is likely to benefit from a treatment regimen that includes a pancreatic beta cell protecting treatment.

Accordingly, the present invention also relates to an in vitro method of determining whether a subject suffering from type 2 diabetes is likely to benefit from a treatment regimen that includes a pancreatic beta cell protecting treatment comprising the steps of:

a) measuring the concentration of bacterial 16S rDNA in a biological sample of said subject as defined in the above section “In vitro method for predicting”; and

b) comparing said measured concentration of bacterial 16S rDNA to a threshold level as defined in the above section “In vitro method for predicting”;

wherein a measured concentration of bacterial 16S rDNA lower than the threshold level indicates that the subject is not likely to benefit from a treatment regimen that includes a pancreatic beta cell protecting treatment.

As used herein, the term “likely to benefit” means that the type 2 diabetes of the subject has an increased probability of being treated as compared to type 2 diabetes of subjects who do not receive a treatment that includes a pancreatic beta cell protecting treatment.

As used herein, the term “treatment regimen” refers to any systematic plan or course for treating a disease in a subject.

In the context of the invention, the expression “pancreatic beta cell protecting treatment” refers to a treatment that specifically targets the protection of pancreatic beta cells. Such treatments is well-known from the skilled person and include for example transplantation of Langherans islets from a healthy donor, in vitro differentiation of embryonic stem cells or induced pluripotent stem cells towards a beta cell fate (as described in Eberhard et al. (2010) Trends in Endocrin. Metab. 21:457-463), treatment with glucagon-like peptide 1 (GLP-1), with mimetics or enhancers of GLP-1 such exenatide, exenatide LAR and liraglutide (as described in Karaca et al. (2009) Diabetes & Metabolism 35:77-84), and with dipeptidyl peptidase-IV (DPP-IV) inhibitors such as sitagliptin phosphate, vildagliptin, metformin, alogliptin benzoate and saxagliptin (as described in Karaca et al. (2009) Diabetes & Metabolism 35:77-84).

The invention will be further illustrated by the following examples and figures.

### DESCRIPTION OF THE FIGURES

FIG. 1 displays graphs representing the distribution of the concentration of 16S rDNA gene, for participants with (dashed line) and without (full line) incident diabetes described in Example 1.

FIG. 2 displays graphs representing the time course of glucose at inclusion and at 3-, 6- and 9-year follow-up visits for the first (), the second (▪), the third (▴) and the fourth (♦) quartiles of 16S rDNA gene concentration in 50-years-old men of the population described in Example 1.

FIG. 3 displays graphs representing the time course of insulin at inclusion and at 3-, 6- and 9-year follow-up visits for the first (), the second (▪), the third (▴) and the fourth (♦) quartiles of 16S rDNA gene concentration in 50-years-old men of the population described in Example 1.

FIG. 4 displays graphs representing the time course of HOMA-beta, which is an index of insulin secretion, at inclusion and at 3-, 6- and 9-year follow-up visits for the first (), the second (▪), the third (▴) and the fourth (♦) quartiles of 16S rDNA gene concentration in men of the population described in Example 1.

FIG. 5 displays graphs representing the time course of HOMA-IR, which is an index of insulin resistance, at inclusion and at 3-, 6- and 9-year follow-up visits for the first (), the second (▪), the third (▴) and the fourth (♦) quartiles of 16S rDNA gene concentration in men of the population described in Example 1.

FIG. 6 displays graphs representing the time course of alanine transferase (ALT), at inclusion and at 3-, 6- and 9-year follow-up visits for the first (), the second (▪), the third (▴) and the fourth (♦) quartiles of 16S rDNA gene concentration in men of the population described in Example 1.

FIG. 7 displays graphs representing the time course of glucose at inclusion and at 3-, 6- and 9-year follow-up visits for the first (), the second (▪), the third (▴) and the fourth (♦) quartiles of 16S rDNA gene concentration in women of the population described in Example 1.

FIG. 8 displays graphs representing the time course of insulin at inclusion and at 3-, 6- and 9-year follow-up visits for the first (), the second (▪), the third (▴) and the fourth (♦) quartiles of 16S rDNA gene concentration in women of the population described in Example 1.

FIG. 9 displays graphs representing the time course of HOMA-beta, which is an index of insulin secretion, at inclusion and at 3-, 6- and 9-year follow-up visits for the first (), the second (▪), the third (▴) and the fourth (♦) quartiles of 16S rDNA gene concentration in women of the population described in Example 1.

FIG. 10 displays graphs representing the time course of HOMA-IR, which is an index of insulin resistance, at inclusion and at 3-, 6- and 9-year follow-up visits for the first (), the second (▪), the third (▴) and the fourth (♦) quartiles of 16S rDNA gene concentration in women of the population described in Example 1.

FIG. 11 displays graphs representing the time course of alanine transferase (ALT), at inclusion and at 3-, 6- and 9-year follow-up visits for the first (), the second (▪), the third (▴) and the fourth (♦) quartiles of 16S rDNA gene concentration in women of the population described in Example 1.

FIG. 12 displays the relation between the fasting glycemia and the logarithm of the baseline 16S rDNA concentration at inclusion () and at 3-year (▪), 6-year (▴) and 9-year (♦) follow-up visits in women of the population described in Example 1.

FIG. 13 displays the relation between the insulin level and the logarithm of the baseline 16S rDNA concentration at inclusion () and at 3-year (▪), 6-year (▴) and 9-year (♦) follow-up visits in women of the population described in Example 1.

FIG. 14 displays the relation between the HOMA-beta level and the logarithm of the baseline 16S rDNA concentration at inclusion () and at 3-year (▪), 6-year (▴) and 9-year (♦) follow-up visits in women of the population described in Example 1.

FIG. 15 displays the relation between the HOMA-IR level and the logarithm of the baseline 16S rDNA concentration at inclusion () and at 3-year (▪), 6-year (▴) and 9-year (♦) follow-up visits in women of the population described in Example 1.

FIG. 16 displays the relation between the ALT level and the logarithm of the baseline 16S rDNA concentration at inclusion () and at 3-year (▪), 6-year (▴) and 9-year (♦) follow-up visits in women of the population described in Example 1.

### EXAMPLES

### Example 1

The following example demonstrates the predictive value of blood bacterial 16S rDNA on the onset of type 2 diabetes.

The incidence of diabetes both in developed and emerging countries has reached epidemic proportions (Shaw et al. (2010) Diabetes Res. Clin. Pract. 87:4-14). A body of evidence demonstrates that the intestinal microbiota, which corresponds to the overall bacterial community present in the intestine, and their corresponding expressed genes, which define the microbiome, play a role in the onset of metabolic disease (Turnbaugh et al. (2006) Nature 444:1027-1031; Turnbaugh et al. (2009) Nature 457:480-484; Cani et al. (2007) Diabetes 56:1761-1772). The causal role of the intestinal microbiota on weight gain was demonstrated in experiments in which germ-free mice colonized with intestinal microbiota from genetically obese ob/ob mice gained more weight than their counterparts colonized with microbiota from lean animals (Turnbaugh et al. (2006) Nature 444:1027-1031). In humans, it was shown that obesity was associated with phylum-level changes in the gut microbiota and reduced bacterial diversity (Turnbaugh et al. (2009) Nature 457:480-484). Furthermore, it has been demonstrated that gut microbiota affects energy balance by influencing the efficiency of calorie harvest from the diet and the way this harvested energy is used and stored (Turnbaugh et al. (2006) Nature 444:1027-1031). In addition, the role of bacterial components within blood in relation to weight and glucose metabolism has also been demonstrated: mice fed normal chow and chronically infused with a low dose of lipopolysaccharides (LPS) developed inflammation, diabetes and obesity whereas mice carrying a deletion in the gene for CD14, a component of the principal receptor for bacterial LPS, did not (Cani et al. (2007) Diabetes 56:1761-1772). Interestingly, in humans, plasma LPS concentrations are increased in apparently healthy subjects eating a high-fat diet (Amar et al. (2008) Am. J. Clin. Nutr. 87:1219-1223).

The inventors herein demonstrated that the presence of bacterial components in blood was one of the initial steps leading to diabetes. It is likely that the whole process takes years or decades. To achieve this demonstration, the inventors investigated whether blood 16S rDNA gene concentration, a specific marker of bacterial presence, could be a marker of the risk of diabetes in a large general population without diabetes.

### Methods

### Population

D.E.S.I.R. is a longitudinal cohort study of 5,212 adults aged 30-65 years at baseline; the primary aim of the study was to describe the natural history of the metabolic syndrome (Fumeron et al. (2004) Diabetes 53:1150-1157). Participants were recruited in 1994-1996 from ten Social Security Health Examination centers in central-western France, from volunteers insured by the French national social security system (80% of the French population—any employed or retired person and their dependents are offered free periodic health examinations). Equal numbers of men and women were recruited in five-year age groups. All participants gave written informed consent, and the study protocol was approved by the CCPPRB (Comité Consultatif de Protection des Personnes pour la Recherche Biomédicale) of the Hôpital Bicêtre (Paris, France). Participants were clinically and biologically evaluated at inclusion and at 3-, 6-, and 9-yearly follow-up visits. The inventors studied individuals without diabetes at baseline (defined by treatment for diabetes or fasting plasma glucose ≧7.0 mmol/l) and those who had a known diabetes status at the 9-year examination, with measurements of baseline 16S rDNA gene concentrations. They excluded those with baseline C reactive protein >30 mg/l.

### Parameters Studied

Weight and height were measured in lightly clad participants and body mass index (BMI) was calculated. Waist circumference, the smallest circumference between the lower ribs and the iliac crests, was also measured. The examining physician noted the family history of diabetes and treatment for diabetes and hypertension were recorded. Hypertension was defined by systolic/diastolic blood pressure of at least 140/90 mmHg or being on antihypertensive medication. Smoking habits were documented in a self-administered questionnaire. The homeostasis model assessments of β-cell function (HOMA-beta) and of insulin resistance (HOMA-IR) (Levy et al. (1998) Diabetes Care 21:2191-2192) were computed using software downloaded at http://www.dtu.ox.ac.uk.

Presence of the metabolic syndrome according to the NCEP criteria (Grundy et al. (2004) Circulation 109:433-438) was recorded. Central adiposity was defined by a waist circumference >102 cm in men and >88 cm in women, high fasting glucose by 6.1 mmol/I, low HOMA-beta by values lower than the first quartile and high HOMA-IR, insulin and alanine transaminase (ALT) by values higher than the third quartile.

### Biological Analyses

Blood was drawn after a 12-h fast. All biochemical measurements except bacterial DNA analysis were from one of four health center laboratories located in France at Blois, Chartres, La Riche, or Orléans. Fasting plasma glucose, measured by the glucose oxidase method, was applied to fluoro-oxalated plasma using a Technicon RA100 (Bayer Diagnostics, Puteaux, France) or a Specific or a Delta device (Konelab, Evry, France). ALT was assayed by different methods: Technicon DAX24, Advia 1650 both from Bayer Diagnostics, a Lab 20, a Specific orf Delta from Konelab, or by an AU400 from Olympus, all by enzymatic method at 37° C. HbA1c was measured by high-performance liquid chromatography, using a L9100 automated ion-exchange analyzer (Hitachi/Merck-VWR, Fontenay-sous-Bois, France) or by DCA 2000 automated immunoassay system (Bayer Diagnostics, Puteaux, France). Both glucose and HbA1c were standardized across laboratories. Insulin was measured centrally by a Micro particle Enzyme Immunoassay with the IMX or the AXSYM automated analyser from Abbott. CRP was assayed by BNII nephelometer (Behring, Rueil Malmaison, France). Total cholesterol and triglycerides were measured by enzymatic methods. Interlaboratory variability was assessed monthly on normal and pathological values.

16S rDNA Gene Concentration Quantification

Total DNA concentration was determined using the Quant-iT™ dsDNA Broad-Range Assay Kit (Invitrogen) and a procedure adapted by the genomic platform of the Genopole Toulouse Midi Pyrénées (http://genomique.genotoul.fr). The mean concentration was 121.1±2.9 ng/μl. Each sample was diluted ten-fold in Tris buffer EDTA. The DNA was amplified by realtime PCR (Stepone+; Applied Biosystems) in optical grade 96-well plates. The PCR reaction was performed in a total volume of 25 μL using the Power SYBR® Green PCR master mix (Applied Biosystems), containing 300 nM of each of the universal forward and reverse primers eubac-F (5′-TCCTACGGGAGGCAGCAGT-3′ (SEQ ID NO: 2)) and eubac-R (5′-GGACTACCAGGGTATCTAATCCTGTT-3′ (SEQ ID NO: 3)). The reaction conditions for amplification of DNA were 95° C. for 10 min and 35 cycles of 95° C. for 15 s and 60° C. for 1 min. The amplification step was followed by a melting curve step according to the manufacturer's instructions (from 60° C. to 90° C.) to determine the specificity of the amplification product obtained. The amount of DNA amplified was compared with a purified 16S rDNA from E. coli BL21 standard curve, obtained by real time PCR from DNA dilutions ranging from 0.001 to 10 ng/μL.

### Outcome

Incident cases of diabetes were identified by treatment for diabetes or a fasting plasma glucose ≧7.0 mmol/l at one of the four three-yearly examinations.

### Statistical Analyses

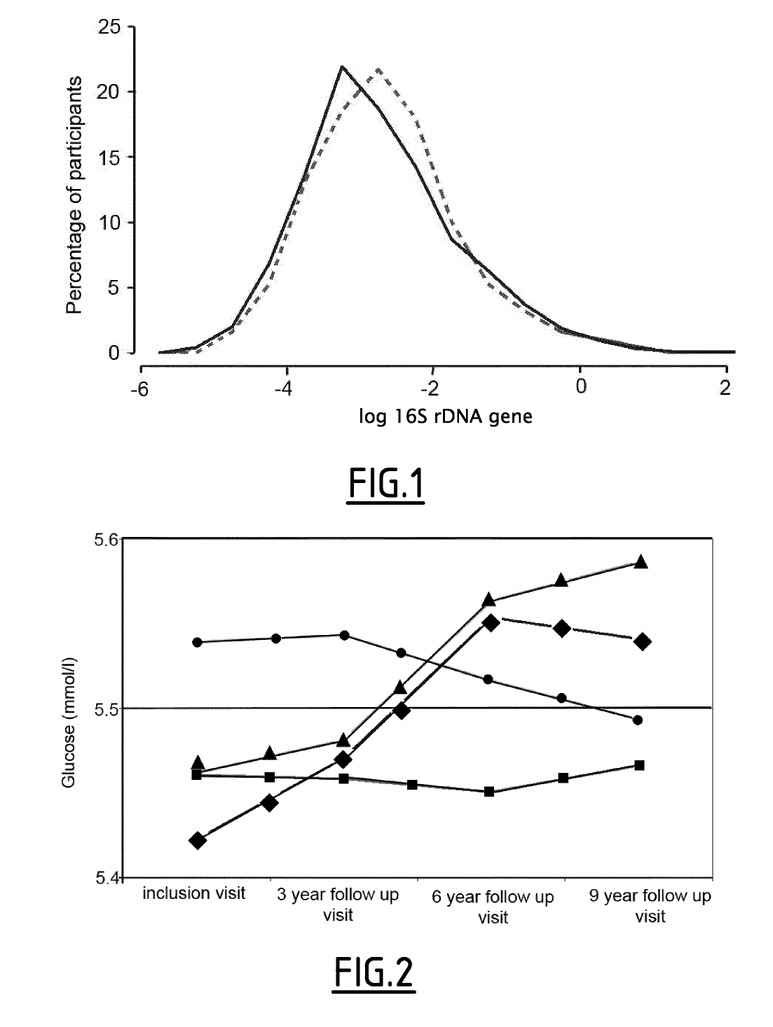

Owing to a skewed distribution (FIG. 1), the 16S rDNA gene concentrations were log transformed, as were the levels of triglycerides, fibrinogen, insulin, C reactive protein (CRP), ALT, HOMA-beta and HOMA-IR. Additionally, 16S rDNA gene concentrations were analyzed in quartile groups. Incident cases of diabetes were analyzed over the entire 9-year follow-up period and then over the three successive time-periods 0 to 3 years, 3 to 6 years and 6 to 9 years, corresponding to the scheduled follow-up visits, as it was hypothesized that the effect of bacteria may take a number of years.

Characteristics of those patients who did and did not become diabetic over the follow-up are shown as means, the standard deviation (SD) being indicated into brackets, or as n, the corresponding percentage in the study population being indicated into brackets. T- and χ²-tests were used to compare those who did and did not become diabetic over the 9 years of follow-up. Baseline characteristics of participants who became diabetic were studied according to the time-interval when they became diabetic, and their characteristics were compared between the three time periods, as well as the trend across time-periods, by analysis of variance and by logistic regression.

Logistic regression was used to calculate the odds ratios and the 95 percent confidence intervals for incident diabetes, over the entire time period, and over the three three-yearly intervals, according to baseline 16S rDNA gene concentrations, as a continuous variable (logarithm, standardized to mean zero, variance 1) and by quartiles with adjustments for sex, baseline age, family history of diabetes, hypertension, waist circumference, BMI, smoking status, fasting plasma glucose. The relation with 16S rDNA gene concentrations (logarithm) was linear, as an additional squared term was not significant. Odds ratios were also calculated over risk-factor strata for the 6 to 9 year period.

The SAS procedure PROC MIXED was used to model repeated data of glucose, insulin, HOMA-beta, HOMA-IR, waist circumference and ALT, over up to four, three-yearly examinations, separately for the quartile groups of 16S rDNA gene concentrations, adjusting for sex and for age. Data were excluded for those years when individuals were treated by drugs for diabetes. Interactions were tested between 16S rDNA gene quartile groups with examination year and sex. The relations for a 50 year old man and for a 50 year old woman are shown graphically as there was a sex interaction. If there was no significant interaction between examination year and 16S rDNA gene concentrations quartile groups, data were modeled and presented graphically, without this interaction. Statistical tests included tests for the linearity of the relation over examination years in each quartile group, and for the difference between quartile groups.

SAS PROC MIXED was also used to model the parameters according to baseline 16S rDNA gene concentrations (logarithm), adjusting for sex, and for age; examination year. The 16S rDNA gene concentrations had an interaction with examination year for all parameters excepting insulin. There was no interaction with gender. The relations for a 50 year old woman are shown graphically; those for a 50 year old man would have the same form but different mean values.

SAS version 9.1 was used for statistical analysis.

### Results

### Studied Population

At baseline, among the 5212 participants in the D.E.S.I.R. study, 126 participants had diabetes, 333 did not undergo 16S rDNA gene concentration determination, two had CRP >30 mg/l and for 1146, diabetes status was not known at the end of the nine years, as they did not attend the 9-year examination. These volunteers were excluded from the analysis. By comparison, the participants analyzed (n=3605) were older and fewer were current smokers. There was no significant difference in baseline 16S rDNA gene concentrations, waist circumference or fasting plasma glucose.

### Characteristics of the Population According to the Year of Incident Diabetes

The risk factors (P<0.05) for diabetes over the nine years of the study were: age, male gender, diabetes in the family, smoking, anthropometry, hypertension and factors associated with inflammation, lipid abnormalities and liver enzymes (Table 1).

TABLE 1
Baseline characteristics (mean (SD) and n (%)) of participants who were and were not screened with incident diabetes during the 9 year follow-up.

| | | Did not | Became | | Became diabetic | | | P value according | |
|---|---|---|---|---|---|---|---|---|---|
| | | become | diabetic over | | Inclusion | 3 to 6 | 6 to 9 | to time of | |
| Baseline | | diabetic | the 9 years | P | to 3 years | years | years | incident diabetes | |
| characteristics | | n = 3416 | n = 189 | value | n = 77 | n = 44 | n = 57 | ANOVA | Trend |
| Age (years) | | 47 (10) | 51 (9) | 0.0001 | 52 (9) | 49 (9) | 51 (9) | 0.4 | 0.5 |
| Women (%) | | 1790 (52%) | 57 (30%) | 0.0001 | 26 (34%) | 10 (23%) | 19 (33%) | 0.4 | 0.9 |
| Diabetes in family | | 638 (19%) | 50 (26%) | 0.01 | 22 (29%) | 15 (34%) | 12 (21%) | 0.3 | 0.4 |
| Current smoker | | 619 (18%) | 55 (29%) | 0.0002 | 19 (25%) | 15 (34%) | 17 (30%) | 0.5 | 0.5 |
| BMI (kg)m²) | | 24.39 (3.44) | 27.99 (4.43) | 0.0001 | 28 (4.49) | 28.66 (5.22) | 27.12 (3.53) | 0.2 | 0.3 |
| Waist circum | men | 88.60 (8.68) | 96.24 (10.17) | 0.0001 | 97.43 (10.50) | 96.21 (10.86) | 93.76 (9.11) | 0.2 | 0.1 |
| (cm) | women | 76.35 (9.62) | 89.58 (11.65) | 0.0001 | 88.65 (11.65) | 96.70 (13.71) | 86.89 (10.03) | 0.09 | 0.7 |

| | | | | | | | | |
|---|---|---|---|---|---|---|---|---|
| Hypertension^{†} | 1151 (34%) | 117 (62%) | 0.0001 | 54 (70%) | 26 (59%) | 30 (53%) | 0.1 | 0.04 |
| Glucose (mmol/l) | 5.24 (0.49) | 6.03 (0.56) | 0.0001 | 6.17 (0.56) | 5.97 (0.57) | 5.87 (0.53) | 0.007 | 0.002 |
| HbA1c (%) | 5.41 (0.38) | 5.83 (0.47) | 0.0001 | 5.94 (0.46) | 5.75 (0.50) | 5.72 (0.44) | 0.01 | 0.005 |
| Insulin (pmol/l)* | 43.8 (24.2) | 71.5 (46.0) | 0.0001 | 67.7 (39.7) | 80.5 (62.4) | 67.8 (40.4) | 0.7 | 0.9 |
| HOMA-beta* | 84.9 (26.8) | 89.2 (42.9) | 1 | 81.9 (37.8) | 98.0 (57.7) | 91.3 (36.7) | 0.1 | 0.07 |
| HOMA-IR* | 1.00 (0.49) | 1.63 (0.97) | 0.0001 | 1.54 (0.86) | 1.82 (1.30) | 1.55 (0.84) | 0.3 | 0.7 |
| Fibrinogen (g/l)* | 2.96 (0.63) | 3.22 (0.72) | 0.0001 | 3.19 (0.66) | 3.17 (0.76) | 3.23 (0.71) | 0.9 | 0.8 |
| CRP (mg/l) | 1.53 (2.40) | 2.38 (2.44) | 0.0001 | 2.62 (3.00) | 2.19 (1.82) | 2.10 (1.75) | 1 | 0.8 |
| Leucocytes (% > 0) | 344 (10%) | 13 (7%) | 0.1 | 8 (10%) | 2 (5%) | 3 (5%) | 0.4 | 0.2 |
| Triglycerides (mmol/l)* | 1.08 (0.66) | 1.71 (1.32) | 0.0001 | 1.65 (1.01) | 2.24 (2.11) | 1.40 (0.76) | 0.006 | 0.2 |
| Total cholesterol (mmol/l) | 5.70 (0.97) | 6.03 (1.06) | 0.0001 | 6.00 (1.05) | 6.19 (1.17) | 5.95 (0.97) | 0.5 | 0.9 |
| LDL-cholesterol (mmol/l) | 3.56 (0.90) | 3.82 (0.91) | 0.0002 | 3.78 (0.90) | 3.85 (0.94) | 3.83 (0.88) | 0.9 | 0.7 |
| ALAT (IU/l)* | 25.0 (17.0) | 38.7 (27.8) | 0.0001 | 38.7 (31.4) | 43.4 (27.4) | 34.9 (24.9) | 0.2 | 0.7 |
| 16S rDNA (ng/μl)* | 0.13 (0.33) | 0.13 (0.21) | 0.4 | 0.12 (0.23) | 0.08 (0.06) | 0.17 (0.26) | 0.04 | 0.04 |

*Logarithms used for analysis
^{†}SBP ≧ 140 mmHg and/or DBP ≧ 90 mmHg and/or an antihypertensive treatment.

Metabolic factors were also associated with risk (P<0.05): glucose, HbA1c, insulin, HOMA-IR, but not HOMA-beta. The mean 16S rDNA gene concentrations were identical (P=0.4) in those with and without incident diabetes, although the distribution was shifted moderately to the right in those with incident diabetes (FIG. 1). Comparing the time-periods when participants became diabetic, hypertension, baseline glucose and HbA1c showed a trend across the three time periods (all P_{trend}<0.05), with the highest values in the first time period. For HOMA-beta, those who became diabetic in the first three year time-period had a lower baseline insulin secretion (P_{trend}=0.07). There was also a significant trend for 16S rDNA gene concentrations, with baseline values highest in those becoming diabetic in the last three year period, those with incident diabetes at year 9 (P_{trend}=0.07). Note that, for 11 participants, the time of diabetes could not be determined, as they did not attend all the examinations. Thus the number of incident diabetic cases indicated at the intermediate years do not sum to the 189 participants with diabetes over the 9 years of the study.

### Prediction of Diabetes

16S rDNA gene concentration predicted the onset of diabetes over the entire nine year follow-up period, after adjustment for confounding factors, in particular baseline fasting plasma glucose, which showed a weakly negative correlation with 16S rDNA gene concentration at baseline (Spearman correlation r=−0.06, P<0.0001) (Table 2).

TABLE 2
Standardized odds ratios (95% confidence intervals) for incident diabetes with an
increase of 1 SD in log (16S rDNA gene concentration)

| | Entire 9 year | | | |
|---|---|---|---|---|
| | follow-up | Inclusion to | 3 to 6 | 6 to 9 |
| | period | 3 years | years | years |
| Number of incident diabetes cases/ | 189/3605 | 77/3486 | 44/3315 | 57/3289 |
| number of participants | | | | |
| Unadjusted ORs (95% CIs) | 1.06 | 0.96 | 0.89 | 1.33 |
| | (0.92-1.23) | (0.76-1.20) | (0.66-1.22) | (1.05-1.70) |
| Number of incident diabetes cases/ | 189/3567 | 77/3466 | 44/3295 | 57/3269 |
| number of participants | | | | |
| Adjusted* ORs (95% CIs) | 1.27 | 1.12 | 0.99 | 1.48 |
| | (1.07-1.50)^{†} | (0.87-1.44) | (0.71-1.38) | (1.15-1.90) |

*Adjusted on sex and baseline age, family history of diabetes, hypertension, smoking status, waist circumference, body mass index and fasting plasma glucose.

For the three three-year time intervals, the 16S rDNA gene concentration was only significantly predictive of incident diabetes in the last time-period, 6 to 9 years, and this remained significant after adjustment for other risk factors, with a standardized odds ratio of 1.48 (95% CI: 1.15-1.90). Similar results were observed for incident diabetes in the 6 to 9 year time period, according to various risk-factor strata: while higher 16S rDNA gene concentrations appeared to carry more risk in men, smokers, those without central adiposity, with lower fasting glucose, ALT, higher HOMA-beta and with lower HOMA-IR and in those without the metabolic syndrome (according to NCEP criteria) (Grundy et al. (2004) Circulation 109:433-438), there was no significant interaction for the risk of incident diabetes between bacterial DNA and any of these strata (Table 3).

TABLE 3
Standardized, adjusted odds ratios (95% confidence intervals) of incident
diabetes between years 6 and 9 of follow-up, for 1 SD of log
(16S rDNA gene concentration) in various strata.

| | | | |
|---|---|---|---|
| Incident | | | |
| diabetes | | | P value: |
| between | | | difference |
| years 6 and 9 | Odds ratio | | between |
| n (%) | (95% CI) | P value | strata |

Sex

| | | | | |
|---|---|---|---|---|
| male | 38 (2.4%) | 1.56 (1.15-2.11) | 0.004 | 0.4 |
| female | 19 (1.1%) | 1.39 (0.86-2.2) | 0.2 | |

Smoking status

| | | | | |
|---|---|---|---|---|
| current smokers | 17 (2.9%) | 1.74 (1.04-2.92) | 0.04 | 0.2 |
| ex smokers | 17 (1.9%) | 1.59 (1.04-2.42) | 0.03 | |
| never smoked | 23 (1.3%) | 1.25 (0.82-1.90) | 0.3 | |

Central adiposity (>102 cm men, >88 cm women)

| | | | | |
|---|---|---|---|---|
| present | 12 (3.8%) | 1.14 (0.67-1.92) | 0.6 | 0.4 |
| absent | 45 (1.5%) | 1.61 (1.21-2.15) | 0.001 | |

High fasting glucose (≧6.1 mmol/l)

| | | | | |
|---|---|---|---|---|
| present | 18 (10.9%) | 1.35 (0.84-2.18) | 0.2 | 0.3 |
| absent | 39 (1.2%) | 1.53 (1.14-2.04) | 0.004 | |

Metabolic syndrome*

| | | | | |
|---|---|---|---|---|
| present | 10 (5.7%) | 1.18 (0.62-2.25) | 0.6 | 0.6 |
| absent | 47 (1.5%) | 1.55 (1.17-2.06) | 0.002 | |

High ALT (≧29.5 UI/l)

| | | | | |
|---|---|---|---|---|
| present | 28 (3.4%) | 1.28 (0.92-1.79) | 0.1 | 0.4 |
| absent | 29 (1.2%) | 1.74 (1.20-2.54) | 0.004 | |

Low HOMA-beta (≦65.9)^{†}

| | | | | |
|---|---|---|---|---|
| present | 11 (1.5%) | 1.27 (0.70-2.30) | 0.4 | 0.3 |
| absent | 46 (1.8%) | 1.59 (1.20-2.10) | 0.001 | |

High HOMA-IR (>0.7)^{†}

| | | | | |
|---|---|---|---|---|
| present | 6 (0.6%) | 0.93 (0.34-2.58) | 0.9 | 0.3 |
| absent | 51 (2.3%) | 1.53 (1.18-1.98) | 0.001 | |

High insulin (≧53.63 pmol/l)^{†}

| | | | | |
|---|---|---|---|---|
| present | 32 (3.9%) | 1.44 (1.02-2.03) | 0.04 | 1 |
| absent | 25 (1%) | 1.54 (1.06-2.24) | 0.02 | |

*Metabolic syndrome defined according to the NCEP criteria.
^{†}Adjusted on sex, baseline age, family history of diabetes, hypertension, waist circumference, smoking status, body mass index and fasting plasma glucose, except when that parameter is being specifically studied.

When 16S rDNA gene concentrations were analyzed in quartiles for the 6-9 years follow-up period, the standardized odds ratios for incident diabetes were 1.58 (0.65-3.84) in quartile 2, 2.12 (0.90-4.98) in quartile 3 and 2.51 (1.10-5.73) in quartile 4. After adjustment for other risk factors, as shown in Table 2, the odds ratios increased to 1.92 (0.76-4.81), 3.50 (1.42-8.62) and 3.63 (1.52-8.70) respectively.

### Time Course of Glucose, Insulin, HOMA-Beta, HOMA-IR, Waist Circumference and ALT

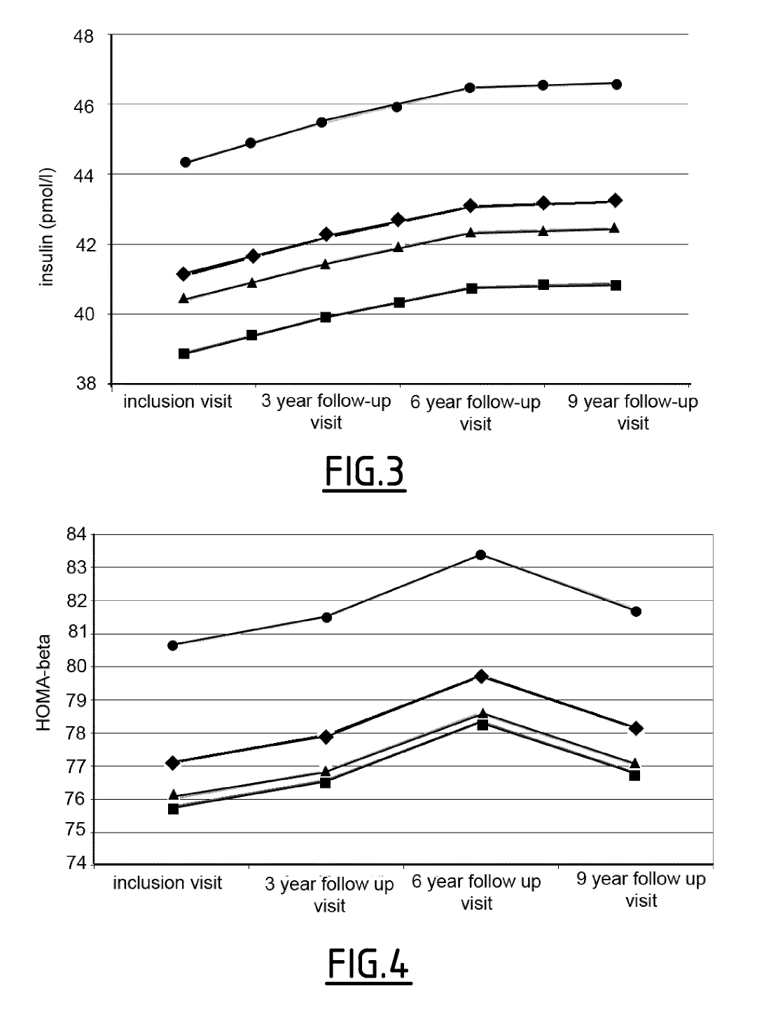

At baseline, in men, glucose levels were lower and insulin levels were higher respectively in the highest and the lowest quartiles of 16S rDNA gene concentrations (FIGS. 2 and 3). Glucose increased over the examinations, only for the upper two quartile groups (P<0.002).

For insulin, HOMA-beta and HOMA-IR, the relation pattern over examination years was the same for all quartile groups. Only the lowest quartile group differed from the other groups (all P<0.01) (FIGS. 4 and 5).

No difference in waist circumference was observed between quartile groups over the follow-up period.

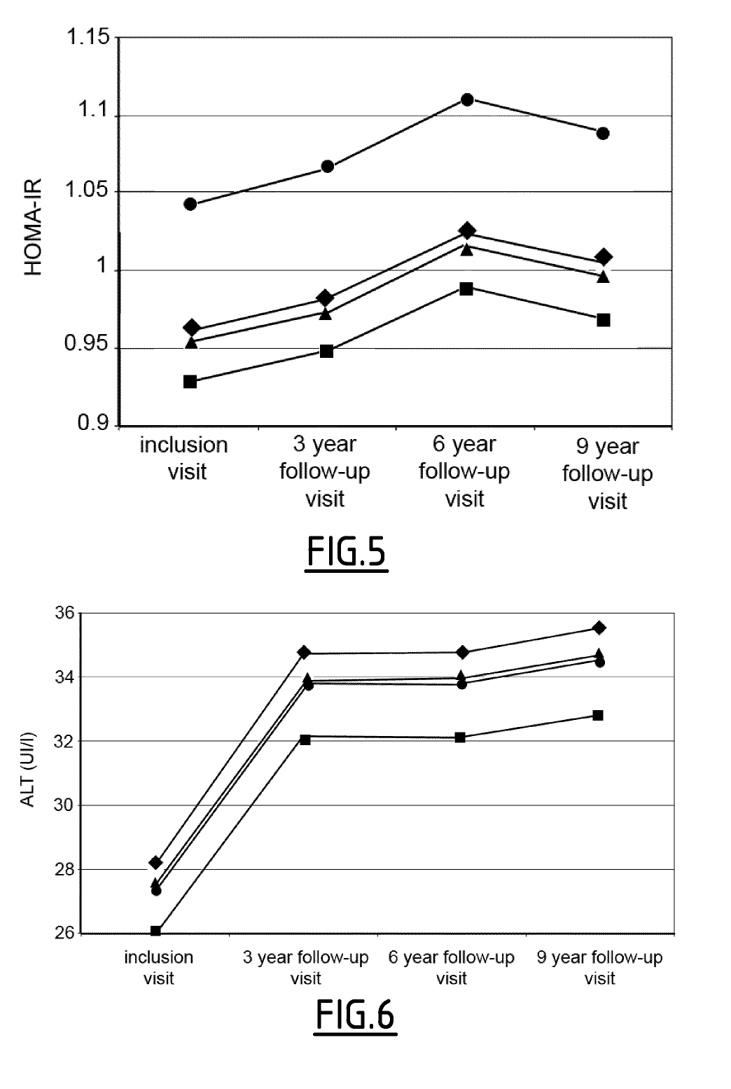

At every examination, ALT was higher in the highest 16S rDNA gene concentration quartile, but only significantly different from the quartile 2 group (P<0.02) (FIG. 6).

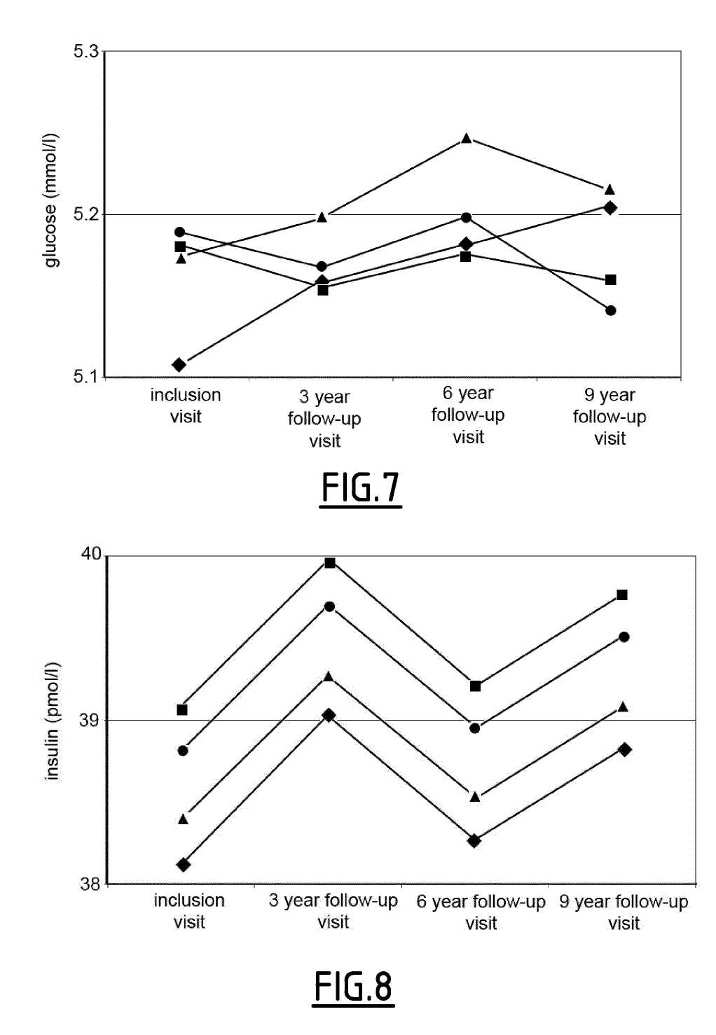

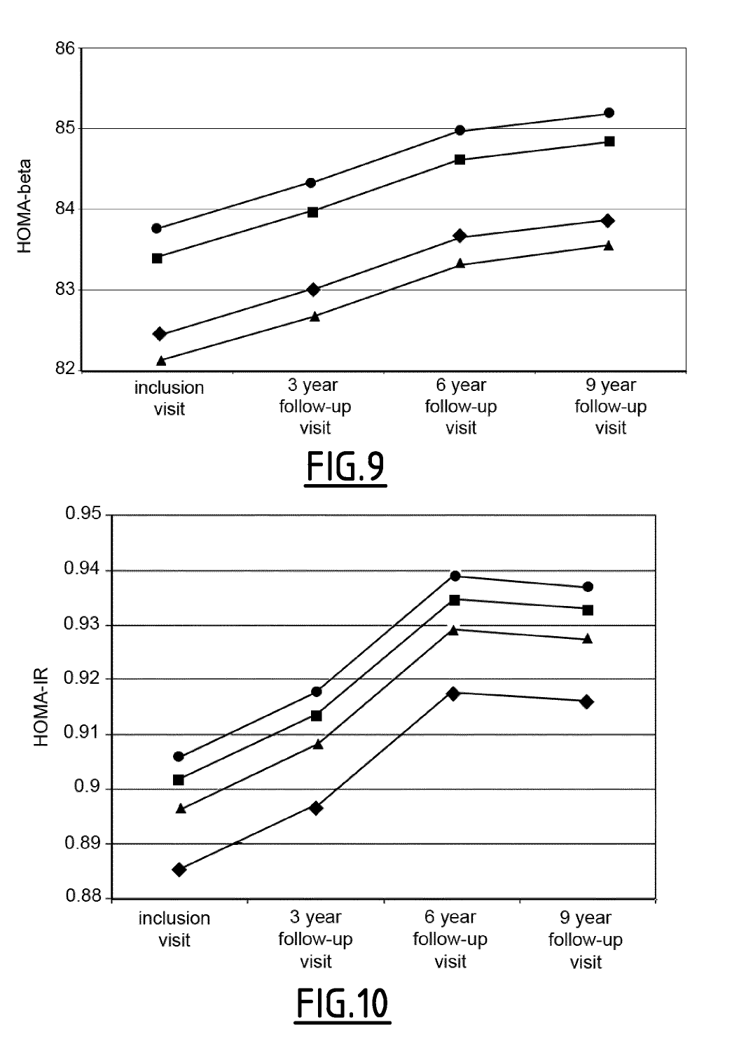

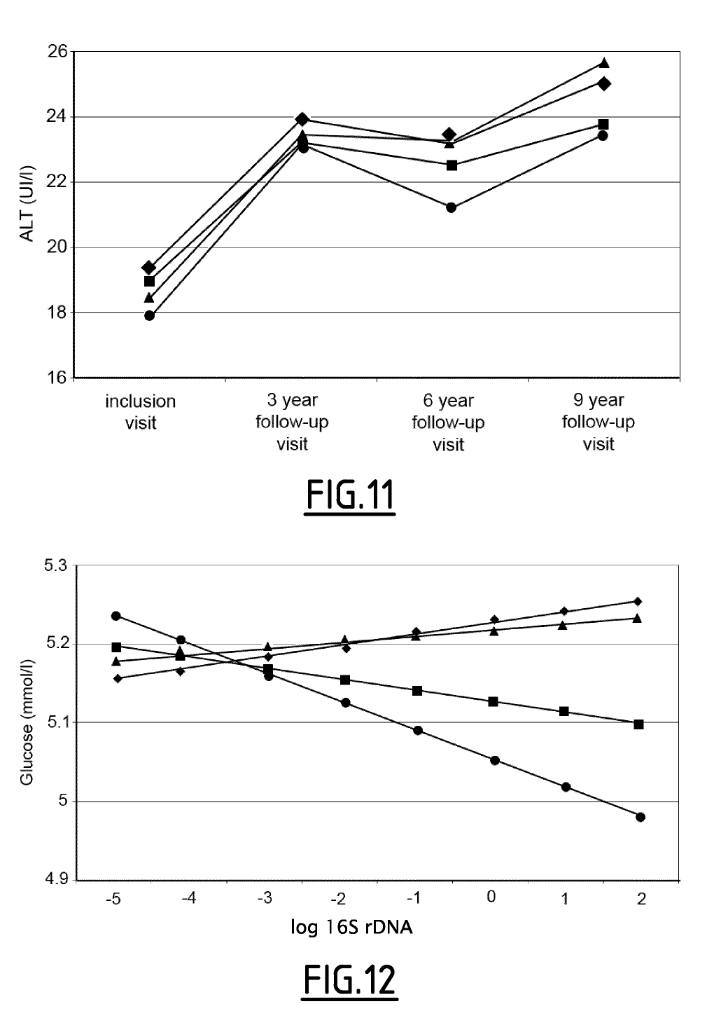

For women (FIGS. 7, 8, 9, 10 and 11), the relation patterns were similar to the ones observed in men. However, the relation between 16S rDNA gene concentration and glucose was the only one to reach statistical significance, with a non-zero increase over time in those in the highest quartile group (P<0.002).

Glucose, Insulin, HOMA-Beta, HOMA-IR, Waist Circumference and ALT According to 16S rDNA Gene Concentrations at Each of the Four Examination.

Since no interaction was observed between gender and 16S rDNA gene concentration and the relations were similar for men and women, typical mean values are only shown for the parameters of 50-year-old women. For men, mean values were higher or lower, but the relation patterns were similar.

At the inclusion examination, glucose (FIG. 12) was negatively associated with 16S rDNA gene concentration (P<0.0001). This relation changed over examination years, and at the final examination, a positive association was observed between baseline 16S rDNA gene concentration and fasting glucose: the higher the concentration of bacterial DNA, the higher the glucose concentration (P<0.01).

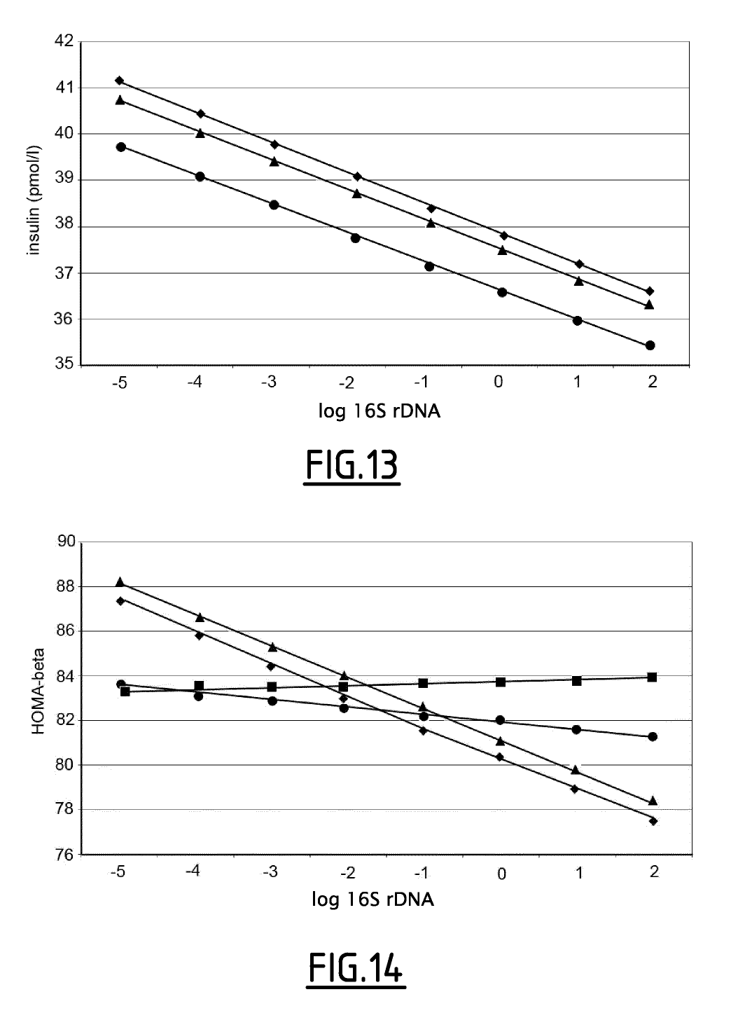

For insulin, the observed correlation was negative (FIG. 13): the higher the 16S rDNA gene concentration, the lower the insulin level, at all examinations (P<0.02). More specifically, the lowest concentrations of insulin, for a given 16S rDNA gene concentration, were observed at the inclusion examination. An identical insulin-bacterial DNA relation was observed at year three and year six examinations and the highest levels were obtained at the final examination. For a given 16S rDNA gene concentration, insulin level was lowest at the inclusion examination and differed from the three later examinations (P<0.01).

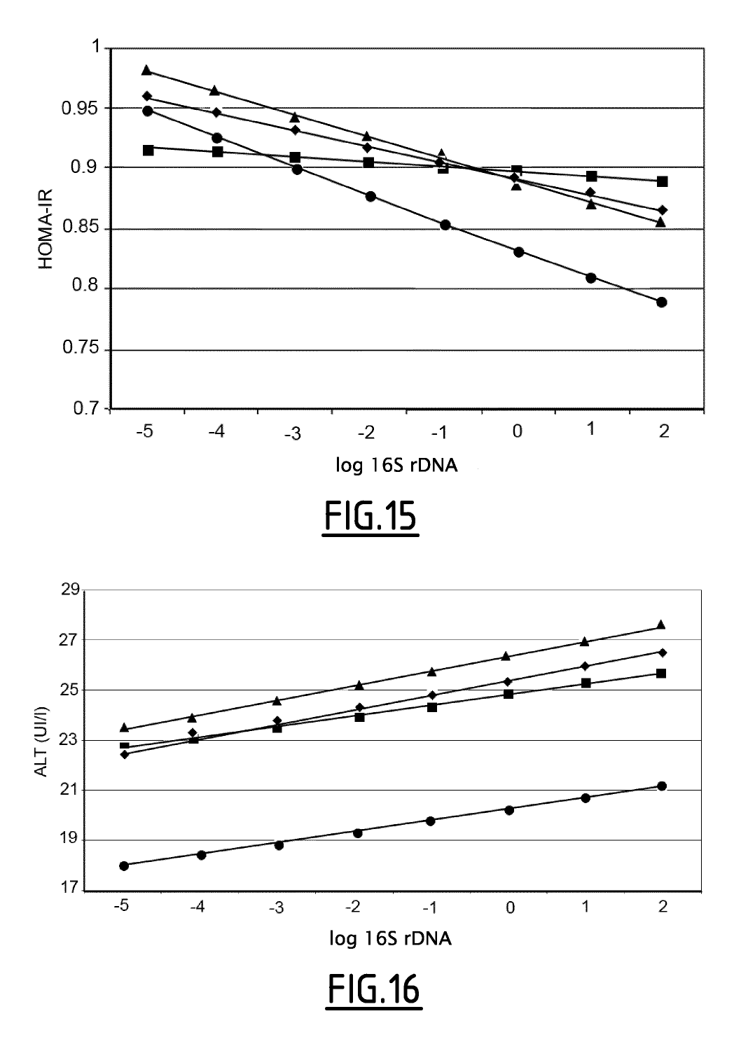

The HOMA-beta index was not significantly associated with the 16S rDNA gene concentration at baseline or at 3-year examination (FIG. 14). However, at year 6 and year 9, the relation was negative: the higher the 16S rDNA gene concentration, the lower the beta cell secretion will be in 6-9 years (both P<0.02). The only significant association for HOMA-IR index was at the inclusion examination where there was a negative association with 16S rDNA gene concentration (P<0.0001) (FIG. 15).

For waist circumference, there was no relation with 16S rDNA gene concentration at any of the examinations, the strongest relation being a negative relation (P=0.07) at inclusion.

ALT was similarly and positively related with 16S rDNA gene concentration at all examinations (all P<0.04) (FIG. 16), indicating a positive association between liver function and bacterial DNA concentrations.

### Discussion

The inventors showed, for the first time, that the concentration of a blood bacterial component, the 16S rDNA gene, predicts the onset of type 2 diabetes in a large sample of apparently healthy subjects, independently of traditional metabolic risk factors. This biomarker is particularly predictive of incident diabetes after a delay of 6-9 years. This predictive value seems to be strengthened in individuals free of central adiposity, high fasting blood glucose or the metabolic syndrome. Further importantly, the inventors could classify the diabetic population by identifying that the bacterial 16S rDNA gene concentration was a predictor of β cell failure.

These new findings have clinical implications. It is predicted that the number of diabetic patients will increase from 285 million adults in 1997 to about 439 million in 2025 (Shaw et al. (2010) Diabetes Res. Clin. Pract. 87:4-14). Therefore, it is of utmost importance to detect those at risk of metabolic diseases at an early stage, when slight lifestyle modifications may be efficient and easier to install. In this respect, currently available risk factors of diabetes, such as central adiposity or high fasting blood glucose, are markers of an advanced stage of metabolic disease. Furthermore, there is a need for a predictive marker independent of metabolic features in order to generate new concepts for therapeutic strategies.

In addition to the predictive value of 16S rDNA gene concentration on the onset of diabetes, the inventors showed that a high concentration of this gene at inclusion was negatively correlated with β cell function at year 6 and year 9 of follow-up. Thus, the inventors provided a marker for the very early prediction of β cell failure which could hence serve as the basis for a preventive treatment where β cells would be better preserved or insulin secretion further enhanced to delay the occurrence of hyperglycemia and hence diabetes. This conclusion makes sense in the light of the recent GLP-1 based therapeutic strategies which aim at restoring a physiological insulin secretion function. Therefore, the very early prediction of β cell failure could be overcome using these new insulin secretion tools.

The longitudinal study reported here provides, for the first time, strong evidence of the role of the microbiome on type 2 diabetes in humans. Furthermore, these epidemiological data provide some insights on the mechanisms of action of the microbiome. First at inclusion, there was a negative correlation between bacterial DNA level and fasting blood glucose and insulin resistance, a relation which reversed over the follow-up. This observation suggests a transitory improvement in insulin sensitivity at the early phase of metabolic infection. Furthermore, there was a negative correlation between 16S rDNA gene concentration and HOMA-beta at 6 and 9 year follow-up. Hence, the inventors' marker predicts β cell failure thereby classifying several years in advance a subset of the future diabetic population i.e. those who will become diabetic due to an early development of β cell insufficiency rather than insulin resistance.

Furthermore, the inventors observed a positive correlation between 16S rDNA gene concentration and alanine transferase throughout follow-up suggesting a negative impact of a subclinical infection on β cell and liver functions.

In conclusion, the inventors showed that bacterial 16S rDNA gene concentration was an early marker of diabetes risk. It further predicts a low β cell capacity which could classify a population at risk of β cell failure-induced diabetes. The inventors provided a new tool for screening populations, independently of a more delayed marker such as fasting blood glucose or even central adiposity. These results reveal the role of the blood microbiome on the onset of diabetes, probably via a deleterious effect on pancreatic β cell function and suggest that the tissue microbiome could be a relevant target to prevent metabolic diseases.

### Example 2

This example further shows the sensitivity, specificity and the negative predictive value of 16S rDNA as a marker of the onset of type 2 diabetes with insulinopenia.

### Methods

### Population

The studied population corresponds to the subjects of the population described in Example 1 which displays at the inclusion at least one diabetes risk factor among
a waist circumference of more than 102 cm in men or of more than 88 cm in women;
smoking;
a fasting glycemia equal or superior to 6.1 mmol/L;
hypertension; and
family history of diabetes.

### Outcome

Type 2 diabetes with insulinopenia was defined as a non-treated diabetes with an HOMA-beta level inferior to the median of the calculated HOMA-beta (using the software downloaded at http://www.dtu.ox.ac.uk) in non-treated subjects, recorded in the DESIR database as diabetic after 6 to 9 years of follow-up.

16S rDNA

The used threshold level of 16S rDNA corresponded to the level of 16S rDNA above the 8^{th} quartile, that is to say 0.15 ng/μl.

### Results

In the conditions described above, the assay applied to a subject at risk of diabetes with regards to traditional risk factors has a very good negative predictive value: a 16S rDNA concentration inferior to 0.15 ng/μl excludes at more than 99% the onset of type 2 diabetes with insulinopenia within 6 to 9 years after the sampling (Table 4), and excludes at more than 97% the onset of type 2 diabetes with insulinopenia within 0 to 9 years after the sampling (Table 5).

TABLE 4
Sensitivity and specificity of 16S rDNA as a predictive marker of the onset
of type 2 diabetes with insulinopenia within 6 to 9 years after sampling.

| Onset of type 2 diabetes with insulinopenia | 16S rDNA ≧8^{th} decile |
|---|---|
| within 6 to 9 years after sampling | (0.15 ng/μl) |
| Negative predictive value | 99.33% |
| Sensitivity | 29.41% |
| Specificity | 80.7% |

TABLE 4
Sensitivity and specificity of 16S rDNA as a predictive marker of the onset
of type 2 diabetes with insulinopenia within 0 to 9 years after sampling.

| Onset of type 2 diabetes with insulinopenia | 16S rDNA ≧8^{th} decile |
|---|---|
| within 0 to 9 years after sampling | (0.15 ng/μl) |
| Negative predictive value | 97.44% |
| Sensitivity | 19.3% |
| Specificity | 80.62% |

Accordingly, the inventors demonstrated that the bacterial 16S rDNA concentration was a potent predictive marker of the non-onset of type 2 diabetes with insulinopenia.

1. An in vitro method for predicting a risk of onset of type 2 diabetes in a subject, which method comprises the steps of:
a) measuring the concentration of bacterial 16S rDNA in a biological sample of said subject; and
b) comparing said measured concentration of bacterial 16S rDNA to a threshold level;
wherein a measured concentration of bacterial 16S rDNA higher than the threshold level is indicative of an increased risk of onset of type 2 diabetes in the subject, and a measured concentration of bacterial 16S rDNA lower than the threshold level is indicative of a decreased risk of onset of type 2 diabetes in the subject.

2. The in vitro method according to claim 1, wherein a measured concentration of bacterial 16S rDNA in the biological sample of the subject which is lower than the threshold level is indicative of a decreased risk of onset of type 2 diabetes with predominant insulinopenia.

3. The in vitro method according to claim 1, wherein a measured concentration of bacterial 16S rDNA higher than the threshold level is further predictive of pancreatic beta cell failure.

4. The in vitro method according to claim 1, wherein a measured concentration of bacterial 16S rDNA in the biological sample of the subject which is lower than the threshold level is indicative of a decreased risk of onset of type 2 diabetes with predominant insulinopenia within 9 years from the sampling.

5. The in vitro method according to claim 4, wherein a measured concentration of bacterial 16S rDNA in the biological sample of the subject which is lower than the threshold level is indicative of a decreased risk of onset of type 2 diabetes with predominant insulinopenia within a period of 6 to 9 years from the sampling.

6. An in vitro method for predicting a risk of hepatic cytolysis in a subject, which method comprises the steps of:
a) measuring the concentration of bacterial 16S rDNA in a biological sample of said subject; and
b) comparing said measured concentration of bacterial 16S rDNA to a threshold level;
wherein a measured concentration of bacterial 16S rDNA higher than the threshold level is indicative of an increased risk of hepatic cytolysis.

7. The in vitro method according to claim 1, wherein the threshold level is 0.15 ng/μL of bacterial 16S rDNA.

8. The in vitro method according to claim 1, wherein the biological sample is selected from the group consisting of blood, serum and plasma sample.

9. The in vitro method according to claim 1, wherein the concentration of bacterial 16S rDNA is measured by real-time PCR.

10. The in vitro method according to claim 1, wherein the subject is at risk of diabetes.

11. The in vitro method according to claim 10, wherein the subject displays at least one diabetes risk factor selected from the group consisting of:
a waist circumference of more than 102 cm in men or of more than 88 cm in women;
smoking;
a fasting glycemia equal or superior to 6.1 mmol/L;
hypertension; and
family history of diabetes.

12. The in vitro method according to claim 1, wherein the subject is free of central adiposity, free of a fasting glycemia equal or superior to 6.1 mmol/L or free of the metabolic syndrome.

13. The in vitro method according to claim 1, wherein the subject is 30-65 years old.

14. The in vitro method according to claim 1, wherein the subject displays a plasma baseline C reactive protein concentration lower than 30 mg/l.

15. An in vitro method of determining whether a subject suffering from type 2 diabetes is likely to benefit from a treatment regimen that includes a pancreatic beta cell protecting treatment comprising the steps of:
a) measuring the concentration of bacterial 16S rDNA in a biological sample of said subject; and
b) comparing said measured concentration of bacterial 16S rDNA to a threshold level;
wherein a measured concentration of bacterial 16S rDNA lower than the threshold level indicates that the subject is not likely to benefit from a treatment regimen that includes a pancreatic beta cell protecting treatment.

16. The in vitro method according to claim 6, wherein the biological sample is selected from the group consisting in blood, serum and plasma sample.

17. The in vitro method according to claim 6, wherein the concentration of bacterial 16S rDNA is measured by real-time PCR.

18. The in vitro method according to claim 6, wherein the subject is at risk of diabetes.

19. The in vitro method according to claim 6, wherein the subject is 30-65 years old.

20. The in vitro method according to claim 6 wherein the subject displays a plasma baseline C reactive protein concentration lower than 30 mg/l.


SEQUENCE LISTING (3 sequences)

SEQ ID NO: 1 - DNA, 1542 bp, Escherichia coli
aaattgaagagtttgatcatggctcagattgaacgctggcggcaggcctaacacatgcaagtcgaacggtaacaggaagcagcttgctgctttgctgacgagtggcggacgggtgagtaatgtctgggaaactgcccgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaagttttcagagatgagaatgtgccttcgggagccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctta

SEQ ID NO: 2 - DNA, 19 bp, Artificial
  Synthetic eubac-F oligonucleotide
tcctacgggaggcagcagt

SEQ ID NO: 3 - DNA, 26 bp, Artificial
  Synthetic eubac-R oligonucleotide
ggactaccagggtatctaatcctgtt